(19) United States
(12) Patent Application Publication
(10) Pub. No.: US 2022/0301694 A1
(43) Pub. Date: Sep. 22, 2022

(54) Method and System for Tracking Inventory and Usage of Pharmaceuticals in Medical Facilities

(71) Applicant: Mark Richard Naples, Dayton, OH (US)

(72) Inventor: Mark Richard Naples, Dayton, OH (US)

(21) Appl. No.: 17/204,921

(22) Filed: Mar. 17, 2021

(51) Int. Cl. G16H 40/20; G16H 20/10; G06F 16/23; G16H 70/40

(52) U.S. Cl. CPC G16H 40/20; G16H 20/10; G06F 16/2379; G16H 70/40; A61J 2205/10

(57) ABSTRACT

Method and system for tracking the inventory and reimbursement status of an inventory of drug doses held at medical facilities and administered to patients are disclosed. In one embodiment, the methods comprise facilitating the addition of drug doses into an inventory database, facilitating the assignment of specific doses to specific patients, and facilitating the transfer of drug doses from one location to another, all accomplished without the necessity of creating and/or scanning machine readable data carriers.

### CROSS-REFERENCE TO RELATED APPLICATION

This application claims the benefit of U.S. Provisional Patent Application No. 62/990,771, filed Mar. 17, 2021, which is incorporated by reference herein in its entirety.

### BACKGROUND OF THE INVENTION

### 1) Field of the Invention

This invention (the “Present Invention” or “PI”) relates to the field of pharmaceutical (vaccines and/or drugs “a drug dose” or “drug doses”) inventory and administration of such to a patient. More specifically, the invention comprises a system and method for the receipt of drug doses into medical offices and clinics (“end user”), assignment of individual doses therein to specific patients, and inter-office transfer of drug doses.

### 2) Description of the Related Art

Under certain conditions resulting from a combination of economic, regulatory, and other external driving forces, end user may choose to keep inventory of multiple different drug doses held in storage in anticipation of administering said drug doses to patients for the treatment of various medical conditions. In many instances group medical practices in various medical specialties have multiple providers, and in addition to a central or main office they also schedule and treat patients in one or more alternate locations (“Remote Location” or “RL”) so that they may conveniently treat large numbers of patients for various medical conditions. In order to maintain accurate drug dose inventory records and in some cases to monitor reimbursement for drug doses previously administered, a medical office may use a current state of the art computerized inventory tracking system (the “TS”). The TS may be configured in such a way as to associate individual patients with specific doses that are administered to the patients, enabling the group practice personnel to maintain records of drug dose administration, track inventory of drug doses across multiple locations, maintain records of inter-office drug dose transfers, monitor reimbursement, and perform other functions. To track each dose individually, the TS may accommodate the scanning of serialized barcodes that are printed on each unit of use by the manufacturer; however, depending on the treatment preferences of the physician there may be a substantial proportion of drug doses used for treatment that do not have a manufacturer provided serialized barcode on the unit of use. In those instances, a printed barcode label or other machine readable data carrier must be affixed to individual drug doses so that each drug dose may be scanned and tracked to enable the TS to perform its intended function. The process of programming and/or printing the machine readable data carrier, in addition to the required scanning of each individual drug dose when receiving inventory, assigning doses, and transferring doses is a cumbersome and time consuming process. The incorporation into the TS of the methods and systems described herein by the PI eliminates or minimizes the impact of these listed shortcomings that are inherent in the TS by eliminating the need to affix identifying labels or tags to individual drug doses.

### BRIEF SUMMARY OF THE INVENTION

The PI is directed to a method comprised of facilitating the receipt of drug doses from a pharmaceutical supplier and subsequent storage by an end user, electronically recording data including drug dose lot number, drug dose expiration date, and product identification number (as approved by the FDA), for subsequent drug dose assignment to patients treated in the central or main office and/or one or more RL where patients are examined and administered drug doses.

The PI is also directed to a method comprised of facilitating the dose assignment to patients treated in the central or main office and/or one or more RL where patients are examined and administered drug doses.

The PI is also directed to a method comprised of the management of a database that records the dose assignment of drug doses from a particular lot with particular patients treated in the central or main office and/or one or more RL where patients are examined and administered drug doses, said method intended to facilitate drug inventory tracking and/or any administrative activity which may become necessary as a result of the acquisition or use of substandard pharmaceutical material, and which further allows the user to track and verify the billing and payment receipt processes through communication with external Electronic Medical Records (“EMR”), Practice Management systems (“PM”) or medical billing systems.

The PI is also directed to a method comprised of the management of inter-office inventory transfer with respect to the TS database, including a method of virtually reassigning drug doses from one location to another and maintaining proper inventory counts of all drug doses in all locations (virtual inter-office inventory transfer).

### BRIEF DESCRIPTION OF THE DRAWINGS

To enable the PI to be easily understood and readily practiced, the PI will now be described for purposes of illustration and not limitation, in connection with the following figures wherein:

FIG. 1 is an illustration of a partial sheet of computer generated printed serialized adhesive drug labels that uniquely identify a particular drug dose and are used to associate specific drug doses with patients who receive the dose.

FIG. 2 is a flowchart of one embodiment of the process as described by the PI wherein drug doses are entered into the inventory database on the Add Inventory screen of the TS.

FIG. 3 is an illustration of one embodiment of an Add Inventory screen of the TS referenced in FIG. 2.

FIG. 4 is a flowchart of one embodiment of the process as described by the PI wherein drug doses are assigned to patients on the Assign Dose screen of the TS.

FIG. 5 is an illustration of one embodiment of an Assign Dose screen of the TS.

FIG. 6 is a flowchart of one embodiment of the process as described by the PI wherein individual drug doses are virtually relocated to other locations within a group practice on the Inventory Adjustment screen of the TS.

FIG. 7 is an illustration of one embodiment of an Inventory Adjustment screen of the TS.

FIG. 8 is an illustration of the Welcome screen of the TS.

### DETAILED DESCRIPTION OF THE INVENTION

The PI will now be described in greater detail by reference to the accompanying drawings in which illustrated embodiments of the invention are provided. The PI may also be embodied in alternate forms not herein described, and should not be construed as limited to the embodiments set forth here; these embodiments are provided so that this disclosure will be thorough and complete, and will fully convey the scope of the invention to those skilled in the art. Like numbers will refer to like elements throughout this document, and to provide clarification on various terms used throughout the text, a glossary follows which is intended to set forth the intended meaning of these terms:

drug assignment—the identification of drug doses that have been or will be administered to particular patients and the recording of such within the TS databases.

virtual—related to activity that occurs within the context of computer software operation, such as manipulating databases or representing places or actions that exist within that context.

machine readable data carrier—any tag or label containing an encoded message that is affixed to a drug dose and subsequently scanned or decoded by a compatible device and decoded for further processing, including but not limited to RFID tags and barcodes of multiple different symbologies.

manufacturer—a drug dose processing concern, performing industrial-scale synthesis of pharmaceutical drugs; or, a drug compounder, a drug dose processing concern that prepares medications according to a unique recipe that combines, mixes, or alters ingredients in order to treat specific illnesses or conditions.

Those skilled in the art will recognize that, while the description of the PI contained herein is focused on pharmaceutical products, the methods and processes described will apply equally to other distributed material. It should also be recognized that as federal guidelines evolve, the methods, systems and processes which are addressed herein will be redefined as necessary to accommodate those new guidelines.

Drug doses received by an end user intended for patient treatment are always identified with a human readable production lot number and an expiration date. In some cases, end users may make use of the TS, through the use of a suitable machine readable data carrier reading device, to enter into the inventory database of the TS each drug dose that has been furnished by a manufacturer or a distributor with a machine readable data carrier containing an identifying product number that usually but not necessarily contains the drug NDC, the production lot number assigned to the drug dose, the drug dose expiration date, and in some cases an encoded serial number. To enter into the inventory database of the TS those drug doses that are received from a manufacturer and do not contain a pre-printed or installed machine readable data carrier, a serialized machine readable data carrier for each drug dose may be created by drug distributor personnel or the end user and be affixed to or maintained in close proximity to each drug dose so that each drug dose received by the end user may be cross checked, through the use of a suitable machine readable data carrier reading device, against the list of doses claimed to have been shipped by the drug dose source. To enter into the inventory database of the TS those drug doses that are received from a manufacturer or distributor and contain a pre-printed or installed machine readable data carrier that does not contain a serialized machine readable data carrier, an end user may choose to create and affix a serialized machine readable data carrier for each drug dose, or alternatively may choose to identify each dose by lot number, such that doses from a specific lot number are not differentiated from one another when entered into the inventory database of the TS.

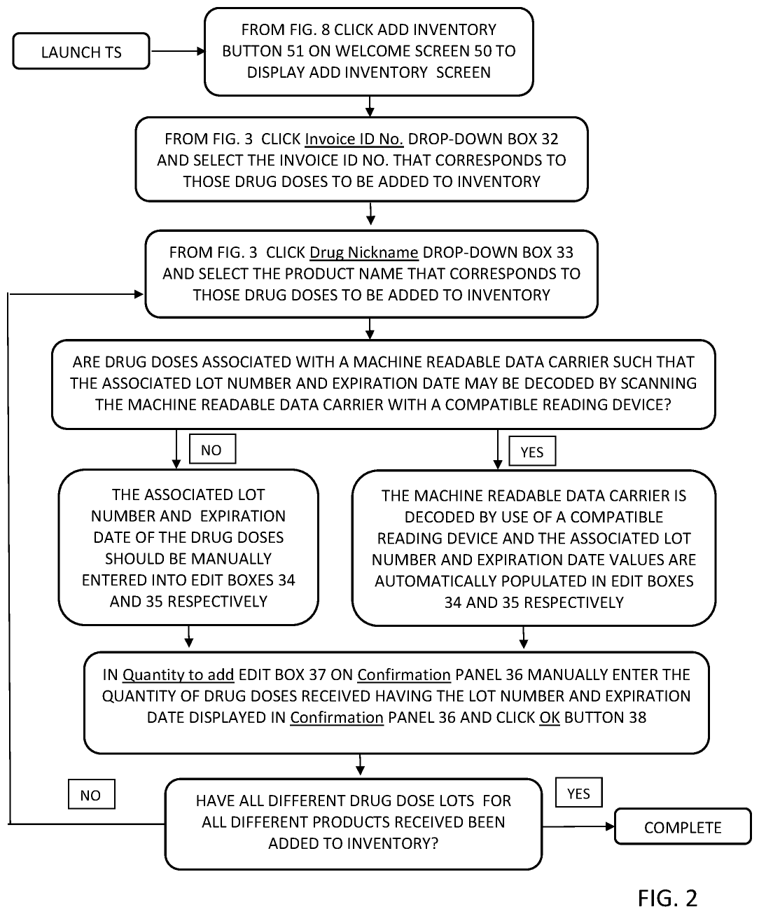

Because an end user may receive several hundred or more drug doses in a day, the PI facilitates the entry of drug doses into the inventory database by introducing the processes detailed in FIG. 2, therefore greatly reducing or eliminating the need to scan and decode a machine readable data carrier:
1) For those drug doses received by the end user that are furnished with a machine readable data carrier either a) containing at least (3) encoded data fields including serial number, lot number, and expiration date, or b) containing at least (2) encoded data fields including lot number, and expiration date, or c) containing at least (1) encoded data field including a unique identification number for the drug dose, and
2) For those drug doses received by the end user that are furnished with no machine readable data carrier.

As drug doses are entered into the inventory database by following the process described in FIG. 2, each individual dose is assigned a virtual serial number by the TS that facilitates the tracking of the dose throughout the lifecycle of the dose as tracked by the TS.

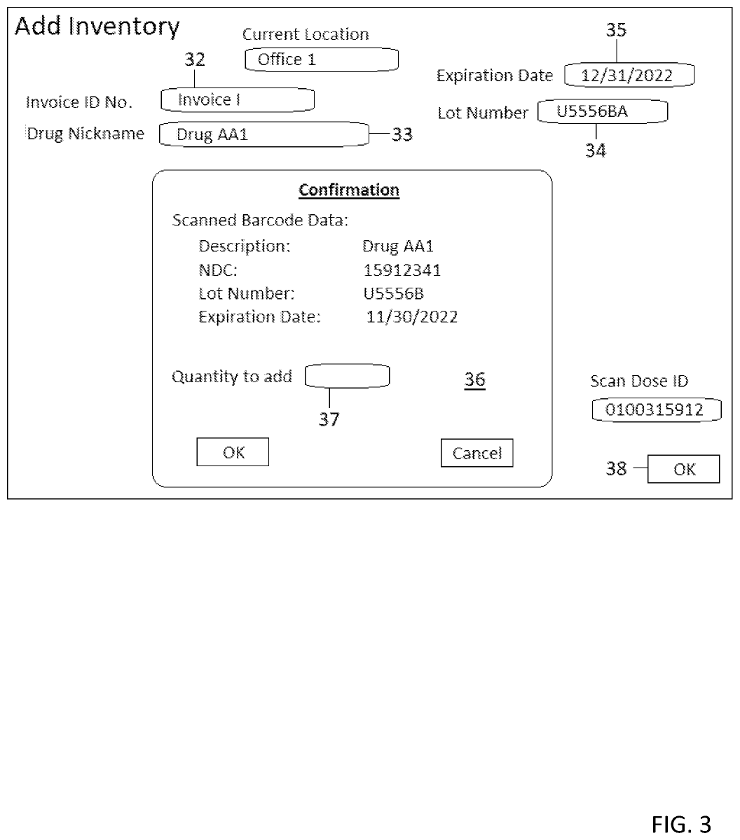

Those skilled in the art will recognize that the process flow and the configuration of the user input screen (the Add Inventory screen) accessed for the entry of drug doses into the inventory database are not limited to the embodiment of the PI described in FIGS. 2 and 3 and that other possible embodiments that describe the PI may exist.

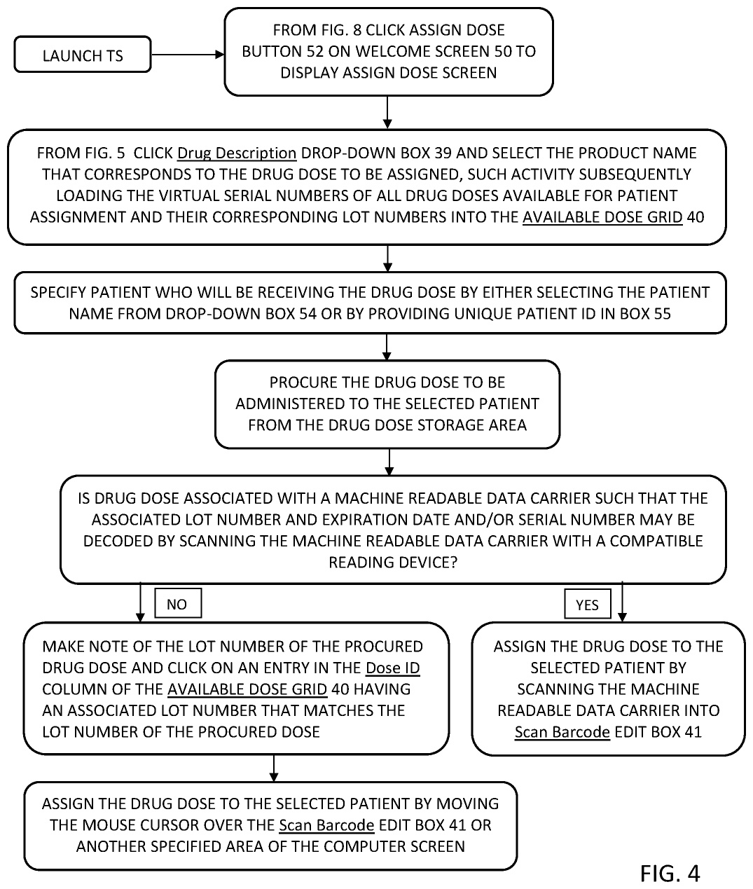

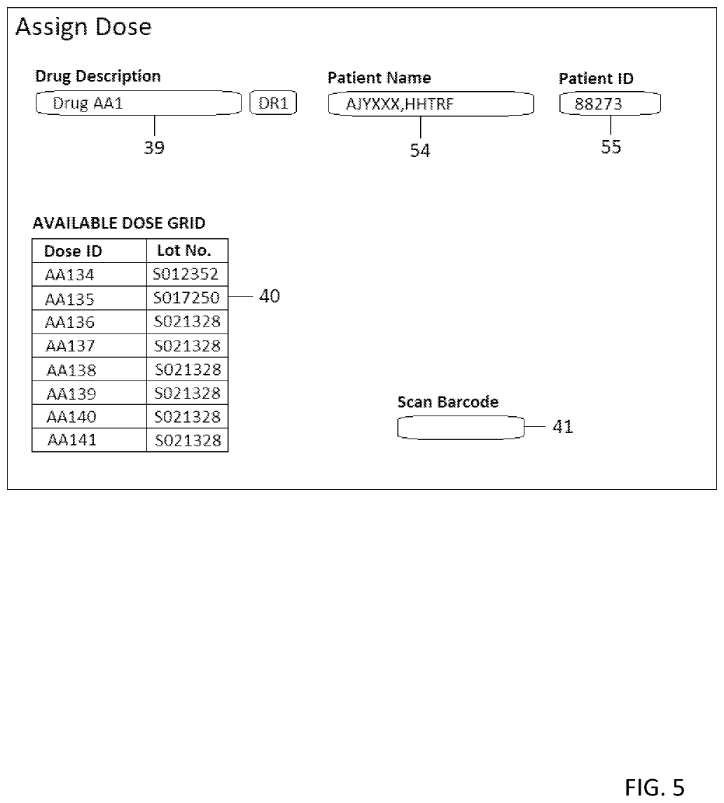

Following receipt of drug doses from a drug dose source and subsequent entry of drug doses into the inventory database, drug doses are administered to patients according to the treatment plan developed by a health care provider. Because a given drug dose selected to be administered to a patient, having previously been entered into the inventory database, may or may not be associated with a machine readable data carrier, the PI describes a general process in FIGS. 4 and 5 that facilitates the assignment of a drug dose that does or does not have an associated machine readable data carrier.

Those skilled in the art will recognize that the process flow and the configuration of the user input screen (the Assign Dose screen) accessed for the assignment of drug doses to patients are not limited to the embodiment of the PI described in FIGS. 4 and 5 and that other possible embodiments that describe the PI may exist.

Because most group medical practices receive and maintain the great majority of pharmaceutical inventory for the entire group at the main or central location, drug doses are generally entered into inventory as having been received and stocked at the main or central location. When patients are to be subsequently examined and treated at one or more RL, drug doses are removed from physical inventory from the main or central office, packed in suitable containers, and shipped or transported to one or more RL for patient treatment. Doses may also be physically relocated among all offices whenever the supply of drug doses warrants such a physical relocation of drug doses.

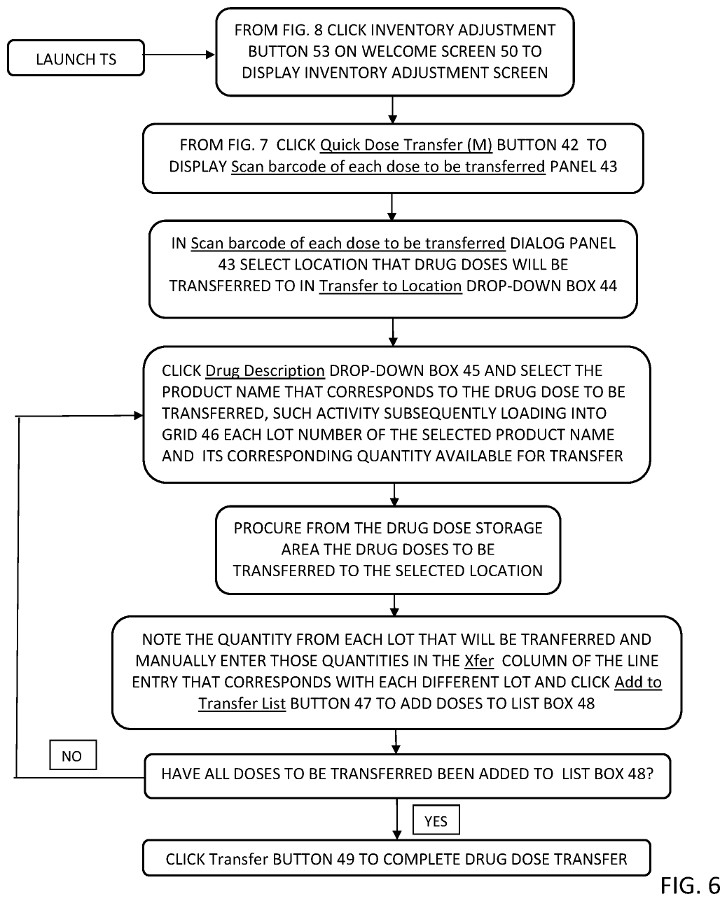

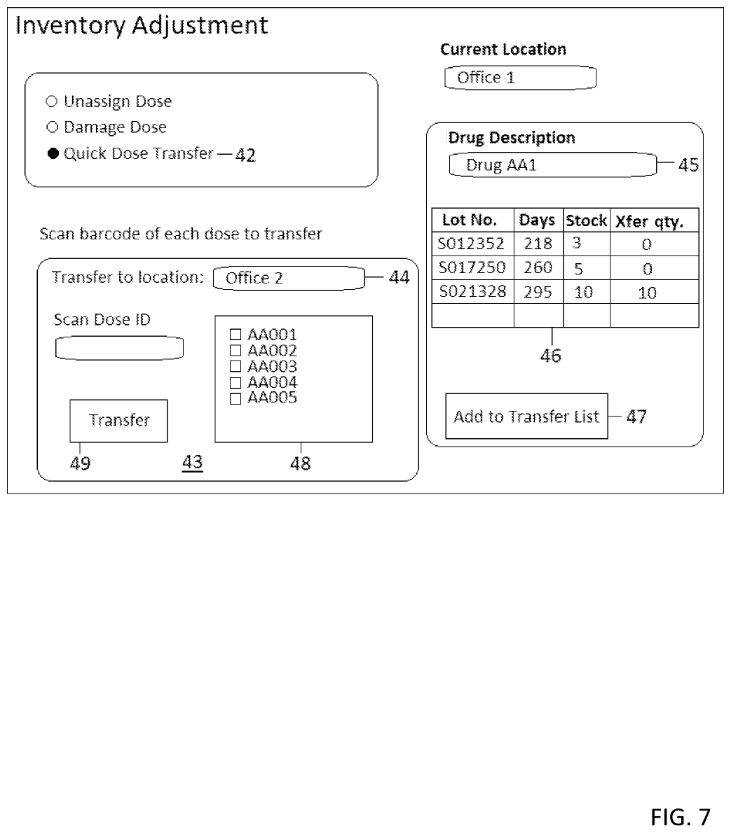

In addition to the physical movement of doses from one location to another, doses must be virtually relocated from the initial inventory location to the RL so that drug dose inventory counts as reported by the TS database remain correct relative to the number of doses of a given drug at a given location. Because drug doses selected to be transferred to an alternate location, having previously been entered into the inventory database, may or may not be associated with a machine readable data carrier, the PI describes a general process in FIGS. 6 and 7 that facilitates the rapid virtual relocation of drug doses with or without a machine readable data carrier encoded with a uniquely identifying serial number. In the case where drug doses are associated with a machine readable data carrier encoded with a uniquely identifying serial number, those doses may be optionally be scanned directly into List Box 48 so that they are added to the queue of drug doses to be transferred.

Those skilled in the art will recognize that the process flow and the configuration of the user input screen (the Inventory Adjustment screen) accessed for the virtual relocation of drug doses are not limited to the embodiment of the PI described in FIGS. 4 and 5 and that other possible embodiments that describe the PI may exist.

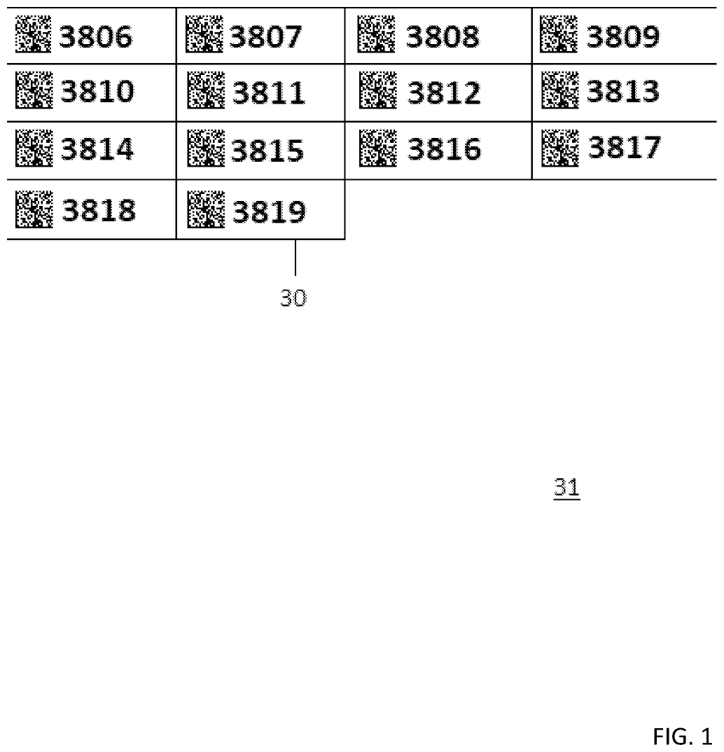

FIG. 1 is an illustration of a partial sheet 31 of computer generated printed serialized adhesive drug labels 30 that uniquely identify a particular drug dose and are used to associate specific drug doses with patients who receive the dose. The PI eliminates the need to create and affix or associate this and all other configurations of machine readable data carriers to drug doses for the purpose of drug dose tracking by end user.

FIG. 2 is a flowchart of one embodiment of the process as described by the PI wherein drug doses are entered into the inventory database on the Add Inventory screen of the TS. In this embodiment, the details of a drug dose shipment that has been received by the end user, including for each different product received, the drug dose product name, labeler, and the quantity received, are received from other data entry modules within the TS and populate the user controls displayed in FIG. 3 that permit the virtual addition of inventory to the database according to the flowchart. Multiple different products may be received in a single drug dose shipment.

FIG. 3 is an illustration of the Add Inventory screen of the TS referenced in FIG. 2.

FIG. 4 is a flowchart of one embodiment of the process as described by the PI wherein drug doses are assigned to patients on the Assign Dose screen of the TS. In the embodiment shown, a listing of available drug doses of the product name selected is displayed within the AVAILABLE DOSE GRID, though a listing of available doses may be communicated to the user through other methods. In addition to the lot number, additional dose specific information may be provided, including but not limited to serial number and expiration date. Other embodiments of the drug dose assignment process may enable drug assignment for those drug doses associated with a machine readable data carrier by clicking a listed available drug dose and moving the cursor to the Scan Barcode edit box, similar to the process described in FIG. 4 for drug doses not associated with a machine readable data carrier.

FIG. 5 is an illustration of the Assign Dose screen of the TS referenced in FIG. 4.

FIG. 6 is a flowchart of one embodiment of the process as described by the PI wherein individual drug doses are virtually relocated to other locations within a group practice on the Inventory Adjustment screen of the TS. In the embodiment shown, a user selects a product name from the Drug Description drop-down box 45 to identify the product to be transferred, though other embodiments may preclude the use of a drop-down box to identify the product to be transferred and instead contain a default product to be transferred, while another embodiment may contain a default product and also include a method for the user to select a product. In addition to the embodiment described in FIG. 6 and FIG. 7 to add drug doses to the drug dose transfer queue displayed in list box 48, those doses associated with a machine readable data carrier containing a serial number may be added to the drug dose transfer queue by scanning with a compatible reading device.

FIG. 7 is an illustration of the Inventory Adjustment screen of the TS referenced in FIG. 6.

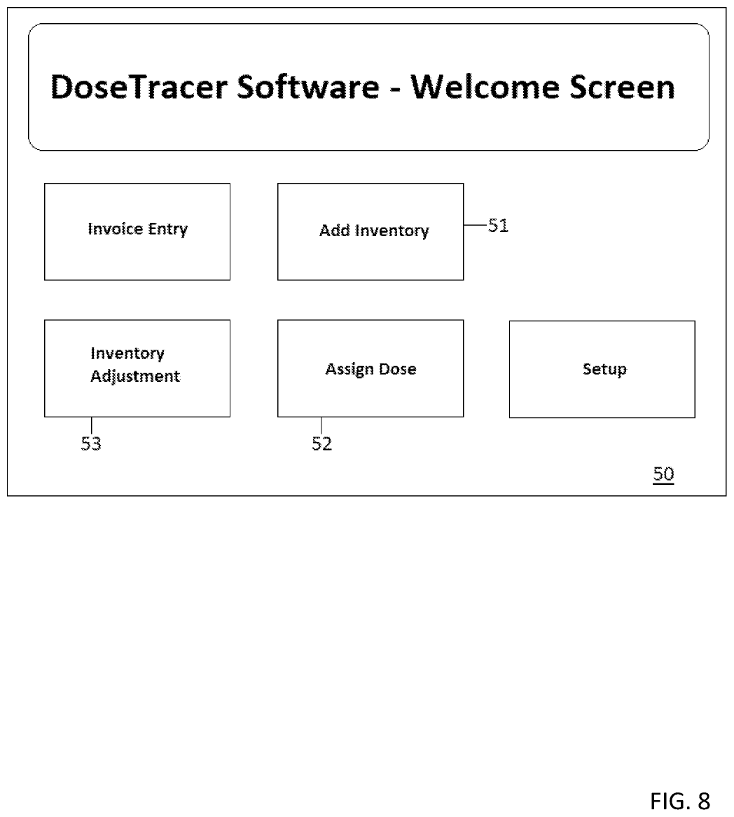

FIG. 8 is an illustration of the Welcome screen 50 of the TS. This is the initial screen displayed after launch of the TS.

What is claimed is:

1. A method comprising:
modifying, in a computerized inventory tracking system, a database comprising inventory counts and identification information of drug doses received by a medical facility from a drug distributor or manufacturer;
modifying, in a computerized inventory tracking system, a database through creation of an association of a specific drug dose with a specific patient; and,
modifying, in a computerized inventory tracking system, a database through revision of inventory counts of drug doses whose inventory location has changed;

2. The method of claim 1, wherein the identification information of drug doses includes a drug dose manufacturing lot number and an expiration date.

3. The method of claim 1, wherein the inventory counts of drug doses are updated by extracting the lot number of an incoming batch of drug doses from a machine readable data carrier associated with the drug dose through the use of a machine readable data carrier reading device and subsequently specifying the quantity received of that lot, or by manually entering the lot number and expiration date of an incoming batch of drug doses and subsequently specifying the quantity received of that lot.

4. The method of claim 1, wherein an association between a drug dose and the patient who receives said drug dose is established by specifying a patient who will be receiving the drug dose and subsequently clicking the unique identifying number of the drug dose from a listing of available drug doses and subsequently moving the cursor of the mouse over a specified area of a computer screen therein establishing the association.

5. The method of claim 1, wherein an association between a drug dose and the patient who receives said drug dose is established by specifying a patient who will be receiving the drug dose and subsequently scanning a machine readable data carrier associated with the drug dose using a machine readable data carrier reading device.

6. The method of claim 1, wherein inventory counts and inventory locations of drug doses may be revised by specifying a quantity of drug doses from available drug doses with a specific lot number to be transferred to a different location.

7. The method of claim 1, wherein inventory counts and inventory locations of drug doses may be revised by scanning a machine readable data carrier encoded with a uniquely identifying serial number.

8. A non-transitory computer readable storage medium comprising instructions which, when executed by a processor, cause the processor to perform steps comprising:
modifying, in a computerized inventory tracking system, a database comprising inventory counts and identification information of drug doses received by a medical facility from a drug distributor or manufacturer;
modifying, in a computerized inventory tracking system, a database through creation of an association of a specific drug dose with a specific patient; and,
modifying, in a computerized inventory tracking system, a database through revision of inventory counts of drug doses whose inventory location has changed;

9. The computer readable storage medium of claim 8, wherein the identification information of drug doses includes a drug dose manufacturing lot number and an expiration date.

10. The computer readable storage medium of claim 8, wherein the inventory counts of drug doses are updated by extracting the lot number of an incoming batch of drug doses from a machine readable data carrier associated with the drug dose through the use of a machine readable data carrier reading device and subsequently specifying the quantity received of that lot, or by manually entering the lot number and expiration date of an incoming batch of drug doses and subsequently specifying the quantity received of that lot.

11. The computer readable storage medium of claim 8, wherein an association between a drug dose and the patient who receives said drug dose is established by specifying a patient who will be receiving the drug dose and subsequently clicking the unique identifying number of the drug dose from a listing of available drug doses and subsequently moving the cursor of the mouse over a specified area of a computer screen therein establishing the association.

12. The computer readable storage medium of claim 8, wherein an association between a drug dose and the patient who receives said drug dose is established by specifying a patient who will be receiving the drug dose and subsequently scanning a machine readable data carrier associated with the drug dose using a machine readable data carrier reading device.

13. The computer readable storage medium of claim 8, wherein inventory counts and inventory locations of drug doses may be revised by specifying a quantity of drug doses from available drug doses with a specific lot number to be transferred to a different location.

14. The computer readable storage medium of claim 8, wherein inventory counts and inventory locations of drug doses may be revised by scanning a machine readable data carrier encoded with a uniquely identifying serial number.

15. A computer system comprising:
a non-transitory computer-readable storage medium storing executable computer program instructions, the computer program instructions comprising instructions that when executed cause a computer processor to perform steps comprising:
modifying a database having inventory counts and identification information of drug doses received by a medical facility from a drug distributor or manufacturer;
modifying a database through creation of an association of a specific drug dose with a specific patient; and,
modifying a database through revision of inventory counts of drug doses whose inventory location has changed;

16. The computer system of claim 15, wherein the identification information of drug doses includes a drug dose manufacturing lot number and an expiration date.

17. The computer system of claim 15, wherein the inventory counts of drug doses are updated by extracting the lot number of an incoming batch of drug doses from a machine readable data carrier associated with the drug dose through the use of a machine readable data carrier reading device and subsequently specifying the quantity received of that lot, or by manually entering the lot number and expiration date of an incoming batch of drug doses and subsequently specifying the quantity received of that lot.

18. The computer system of claim 15, wherein an association between a drug dose and the patient who receives said drug dose is established by specifying a patient who will be receiving the drug dose and subsequently clicking the unique identifying number of the drug dose from a listing of available drug doses and subsequently moving the cursor of the mouse over a specified area of a computer screen therein establishing the association.

19. The computer system of claim 15, wherein an association between a drug dose and the patient who receives said drug dose is established by specifying a patient who will be receiving the drug dose and subsequently scanning a machine readable data carrier associated with the drug dose using a machine readable data carrier reading device.

20. The computer system of claim 15, wherein inventory counts and inventory locations of drug doses may be revised by specifying a quantity of drug doses from available drug doses with a specific lot number to be transferred to a different location.
